(19) United States
(12) Patent Application Publication
(10) Pub. No.: US 2019/0335688 A1
(43) Pub. Date: Nov. 7, 2019

(54) METHODS AND SYSTEMS FOR OPTIMIZING AND MONITORING GROUNDWATER AND SOLAR ENERGY USAGE

(71) Applicant: INTERNATIONAL BUSINESS MACHINES CORPORATION, Armonk, NY (US)

(72) Inventors: Seshu TIRUPATHI, Dublin (IE); Francesco FUSCO, Maynooth (IE); Sean A. MCKENNA, Blanchardstown (IE)

(73) Assignee: INTERNATIONAL BUSINESS MACHINES CORPORATION, Armonk, NY (US)

(21) Appl. No.: 15/971,040

(22) Filed: May 4, 2018

(51) Int. Cl. A01G 25/16; G01W 1/10; F04B 13/00; G01W 1/12; G01N 33/24; G01W 1/14; G06N 99/00; G01J 1/42

(52) U.S. Cl. CPC A01G 25/16; G01W 1/10; F04B 13/00; G01W 1/12; G01N 33/24; G01J 2001/4266; G06N 99/005; G01J 1/42; G01W 2203/00; G01N 2033/245; G01W 1/14

(57) ABSTRACT

Embodiments for groundwater and solar energy usage optimization for an agricultural region in an Internet of Things (IoT) computing environment by one or more processors are described. An amount of water required for an agricultural region and an amount of solar energy required to pump the water in a water pumping system for the agricultural region may be determined according to groundwater characteristics, weather data, weather forecasts, solar energy forecasts, historical water pumping data, crop and soil characteristics, agricultural management strategies, or a combination thereof.

### BACKGROUND OF THE INVENTION

### Field of the Invention

The present invention relates in general to computing systems, and more particularly, to various embodiments for monitoring and optimizing groundwater and solar energy usage in an agricultural region.

### Description of the Related Art

Approximately 0.8% of the total water on earth is in the form of fresh groundwater, which is largely responsible for meeting the needs of humans on a daily basis. As such, fresh groundwater is a highly constrained resource. Monitoring the usage of groundwater (and/or preventing groundwater theft or over-discharge) is a critical challenge considering the ever-increasing demand for fresh water and how easily it may be accessed. However, regulating the usage and ensuring that only the required amount of water for a selected region (e.g., a farm) at selected periods of time is abstracted is a key challenge. Such regulation and abstraction becomes more critical for groundwater abstraction powered by solar energy.

### SUMMARY OF THE INVENTION

Various embodiments for monitoring and optimizing groundwater and solar energy usage by one or more processors are described. In one embodiment, by way of example only, a method for monitoring and optimizing groundwater and solar energy usage optimization for an agricultural region in an Internet of Things (IoT) computing environment, again by one or more processors, is provided. An amount of water required for an agricultural region and an amount of solar energy required to pump the water in a water pumping system for the agricultural region may be determined according to groundwater characteristics, weather data, weather forecasts, solar energy forecasts, historical water pumping data, crop and soil characteristics, agricultural management strategies, or a combination thereof.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a block diagram depicting an exemplary computing node according to an embodiment of the present invention;

FIG. 2 is an additional block diagram depicting an exemplary cloud computing environment according to an embodiment of the present invention;

FIG. 3 is an additional block diagram depicting abstraction model layers according to an embodiment of the present invention;

FIGS. 5-6 are diagrams illustrating certain aspects of functionality according to the present invention; and

FIG. 7 is a flowchart diagram depicting an exemplary method for monitoring groundwater discharge in which various aspects of the present invention may be implemented.

### DETAILED DESCRIPTION OF THE DRAWINGS

As previously indicated, groundwater is an important water resource for agricultural regions (e.g., farms), especially in developing countries. Pumping water traditionally has been an expensive option for agricultural regions such as, for examples, farmers. The usage of solar energy for water pumping has significantly reduced the cost for groundwater abstraction. However, this has resulted in a bigger problem of over-exploiting groundwater resource and abstracting more water than required. Moreover, assessing the amount of water to be used in agricultural regions is based solely on educated guesses. Furthermore, current operations fail to provide incentives for selling back harvested solar energy back to a power grid in conjunction with monitoring the amount of solar energy for use along with the amount of water required in the agricultural region. In view of the foregoing, a need exists for methods and systems that monitor and optimize groundwater and solar energy usage in an agricultural region.

To address these needs, the methods and systems of the present invention utilize, for example, analytical and computational techniques along with sensor data to develop quantitative measures for providing a trade-off between groundwater required for an agricultural region and selling excess energy back to the power grid and/or using excess water for storing and non-irrigation activities. Analytical, physical and numerical operations, and machine learning operations, along with sensor data, may be used to predict quantitative measures of water usage and the amount of energy that can be sold back to the power grid. One or more sensors (e.g., an Internet of Things “IoT” sensor device) may be required for the various models for predicting water usage and solar energy generation.

With respect to the following description, “licensed discharge” may refer to a pumping rate (or the amount of groundwater used) approved by the license-issuing authority regulating the groundwater usage at a particular location (e.g., a local government). “Reference head” may refer to a height (or “head”) of groundwater at a particular location in a region (e.g., an agricultural region), or just outside the region, that provides the average groundwater level in that region. “Groundwater head” may refer to a height to which groundwater has risen, at a particular location, above a reference plane (e.g., the reference head). “Radius of influence” may refer to the distance from a particular location up to which groundwater flow is influenced by the groundwater at the particular location.

For example, in some embodiments, a system is provided that enables a quantifiable way of determining if the groundwater usage (or discharge) at a particular location, or multiple locations, such as wells, is greater than the licensed value for a quasi-steady state aquifer. The system may store the discharge limit and the coordinates for wells in a given region through information from the license permits. Groundwater heads measured by sensors, either in a particular well of interest or nearby observation wells (or locations) within the radius of influence, may be recorded and used for analysis. The reference head for the region may also be recorded. The system may also record estimates of various characteristics of the region related to groundwater, such as hydraulic conductivity, transmissivity, aquifer depth, river flow rates, and permeabilities.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based email). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

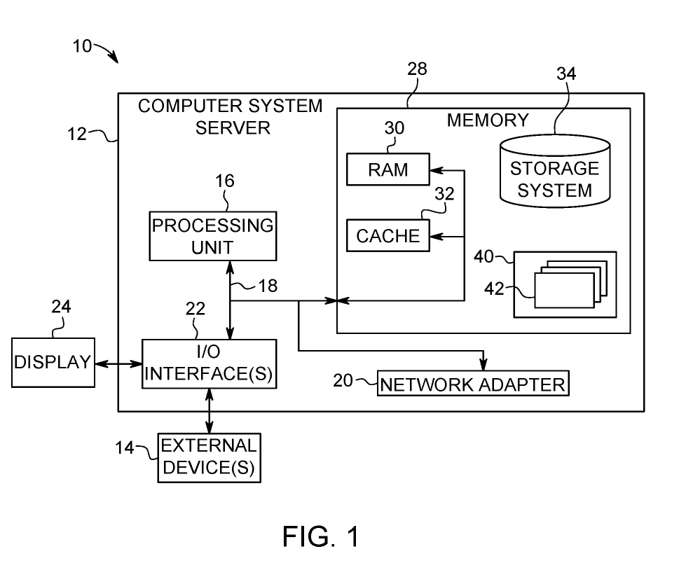

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 (and/or one or more processors described herein) is capable of being implemented and/or performing (or causing or enabling) any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, system memory 28 may include at least one program product having a set (e.g., at least one) of program modules that are configured to carry out the functions of embodiments of the invention.

Program/utility 40, having a set (at least one) of program modules 42, may be stored in system memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples include, but are not limited to: microcode, device drivers, redundant processing units, external disk drive arrays, RAID systems, tape drives, and data archival storage systems, etc.

In the context of the present invention, and as one of skill in the art will appreciate, various components depicted in FIG. 1 may be located in, for example, personal computer systems, hand-held or laptop devices, and network PCs. However, in some embodiments, some of the components depicted in FIG. 1 may be located in a computing device in, or associated with, a groundwater sensor. For example, some of the processing and data storage capabilities associated with mechanisms of the illustrated embodiments may take place locally via local processing components, while the same components are connected via a network to remotely located, distributed computing data processing and storage components to accomplish various purposes of the present invention. Again, as will be appreciated by one of ordinary skill in the art, the present illustration is intended to convey only a subset of what may be an entire connected network of distributed computing components that accomplish various inventive aspects collectively.

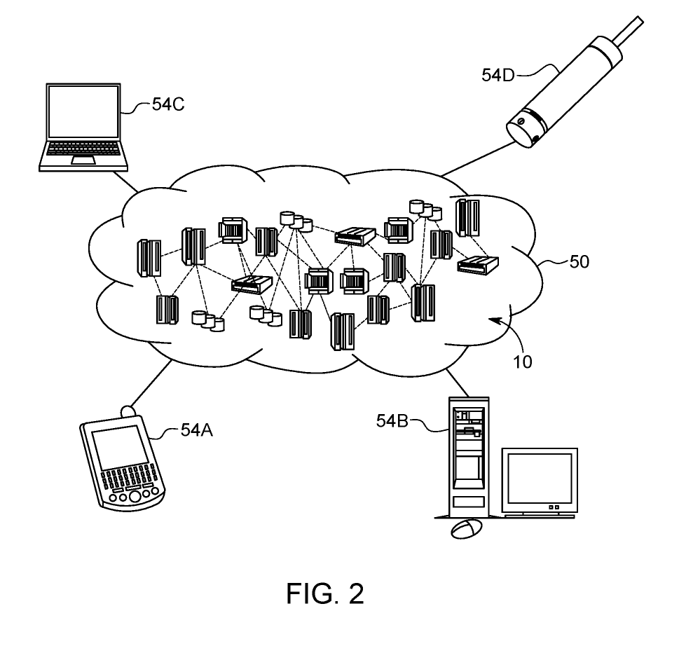

Referring now to FIG. 2, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, and/or laptop computer 54C, and others computer systems, such as, for example, those in, or associated with, groundwater and/or solar energy sensors 54D, may communicate. The groundwater and/or solar energy sensors 54D may include, for example, water level sensors, such as pressure transducers (e.g., piezometers), bubblers, shaft encoders, or ultrasonic sensors, and sensors suitable for measuring other characteristics related to groundwater, such as hydraulic conductivity, transmissivity, aquifer depth, river flow rates, and permeabilities. The groundwater and/or solar energy sensors 54D may also be photovoltaics (PV) sensors.

Still referring to FIG. 2, nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-D shown in FIG. 2 are intended to be illustrative only and that computing nodes 10 and cloud computing environment 50 can communicate with any type of computerized device over any type of network and/or network addressable connection (e.g., using a web browser).

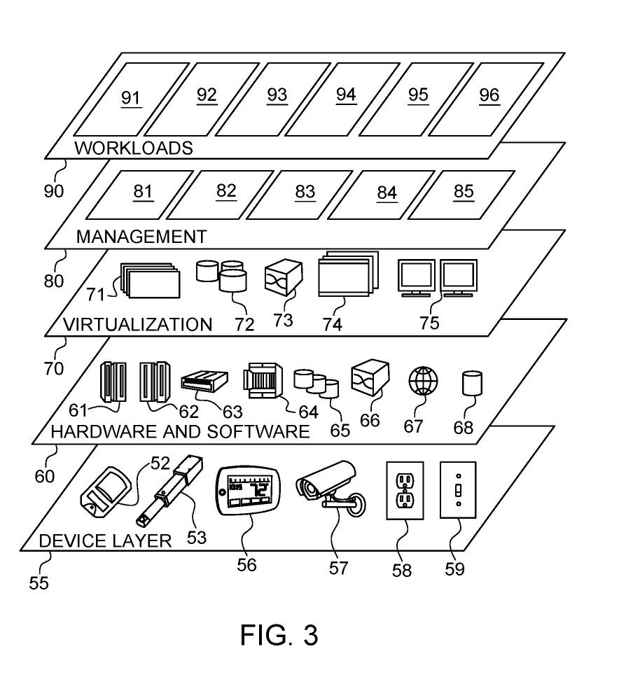

Referring now to FIG. 3, a set of functional abstraction layers provided by cloud computing environment 50 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Device layer 55 includes physical and/or virtual devices, embedded with and/or standalone electronics, sensors, actuators, and other objects to perform various tasks in a cloud computing environment 50. Each of the devices in the device layer 55 incorporates networking capability to other functional abstraction layers such that information obtained from the devices may be provided thereto, and/or information from the other abstraction layers may be provided to the devices. In one embodiment, the various devices inclusive of the device layer 55 may incorporate a network of entities collectively known as the “internet of things” (IoT). Such a network of entities allows for intercommunication, collection, and dissemination of data to accomplish a great variety of purposes, as one of ordinary skill in the art will appreciate.

Device layer 55 as shown includes sensor 52, actuator 53, “learning” thermostat 56 with integrated processing, sensor, and networking electronics, camera 57, controllable household outlet/receptacle 58, and controllable electrical switch 59 as shown. Other possible devices may include, but are not limited to various groundwater sensors, and various additional sensor devices, networking devices, electronics devices (such as a remote control device), additional actuator devices, so called “smart” appliances such as a refrigerator or washer/dryer, and a wide variety of other possible interconnected objects.

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provides cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provides pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and, in the context of the illustrated embodiments of the present invention, various workloads and functions 96 for monitoring and optimizing groundwater and solar energy usage as described herein. One of ordinary skill in the art will appreciate that the monitoring and optimizing groundwater and solar energy usage workloads and functions 96 may also work in conjunction with other portions of the various abstractions layers, such as those in hardware and software 60, virtualization 70, management 80, and other workloads 90 (such as data analytics processing 94, for example) to accomplish the various purposes of the illustrated embodiments of the present invention.

As previously mentioned, the methods and systems of the illustrated embodiments provide novel approaches for monitoring and optimizing groundwater and solar energy usage. In particular, in some embodiments, methods and systems are provided for using groundwater characteristics, historical weather data and weather forecasts, crop and soil characteristics, historical pumping data and other farm management strategies to compute the amount of water required and corresponding photovoltaics (PV) energy required to pump water such as, for example, in a water pumping system that uses PV energy.

Turning now to FIG. 4, a block diagram depicting exemplary functional components 400 according to various mechanisms of the illustrated embodiments is shown. FIG. 4 illustrates cognitive data curation workloads and functions and training of a machine-learning model in a computing environment, such as a computing environment 402, according to an example of the present technology. As will be seen, many of the functional blocks may also be considered “modules” or “components” of functionality, in the same descriptive sense as has been previously described in FIGS. 1-4. With the foregoing in mind, the module/component blocks 400 may also be incorporated into various hardware and software components of a system in accordance with the present invention. Many of the functional blocks 400 may execute as background processes on various components, either in distributed computing components, or on the user device, or elsewhere. Computer system/server 12 is again shown, incorporating processing unit 16 and memory 28 to perform various computational, data processing and other functionality in accordance with various aspects of the present invention.

The system 400 may include the computing environment 402, a water and solar energy usage optimization system 430, one or more IoT devices 450 (e.g., IoT sensor devices), and one or more devices such as, for example device 420 (e.g., a desktop computer, laptop computer, tablet, smartphone, and/or another electronic device that may have one or more processors and memory). The device 420, the IoT devices 450, the water and solar energy usage optimization system 430, and the computing environment 402 may each be associated with and/or in communication with each other, by one or more communication methods, such as a computing network. In one example, the device 420, the IoT devices 450, and/or the water and solar energy usage optimization system 430 may be controlled by an owner, customer, or technician/administrator associated with the computing environment 402. In another example, the device 420, the IoT devices 450, and/or the water and solar energy usage optimization system 430 may be completely independent from the owner, customer, or user of the computing environment 402. The IoT devices 450 may also be associated with a PV energy water pump 475 (e.g., PV energy water pump system). The PV energy water pump 475 may also be in communication with the computing environment 402.

In one aspect, the computing environment 402 may provide virtualized computing services (i.e., virtualized computing, virtualized storage, virtualized networking, etc.) to device 420 and/or the IoT devices 450. More specifically, the computing environment 402 may provide virtualized computing, virtualized storage, virtualized networking and other virtualized services that are executing on a hardware substrate.

As depicted in FIG. 4, the computing environment 402 may include a machine learning component 406, a knowledge domain component 404 that is associated with the machine learning component 406, and the water and solar energy usage optimization system 430. The knowledge domain component 404 may also include an ontology, knowledge base, and/or other data for the water and solar energy usage optimization system 430 and/or associated with IoT devices 450. For example, the ontology and/or knowledge base may include information such as, for example, groundwater characteristics, weather data, weather forecasts, solar energy forecasts, historical water pumping data, crop and soil characteristics, agricultural management strategies, and/or other data.

The knowledge domain component 404 may be a combination of concepts, relationships between the concepts, machine learning data, features, parameters, data, profile data, historical data, models (e.g., weather forecast models, crop/agricultural models, solar energy forecast models, ground water models, etc.), tested and validated data, or other specified/defined data for testing, monitoring, validating, detecting, learning, analyzing, monitoring, and/or maintaining data, concepts, and/or relationships between the concepts in the water and solar energy usage optimization system 430.

The computing environment 402 may also include a computer system 12, as depicted in FIG. 1. The computer system 12 may also include a supply and demand forecast component 410, an integrator component 440, and/or a market connector component 445 each associated with the machine learning component 406 for training and learning one or more machine learning models and also for applying inferences and/or reasoning pertaining to one or more weather forecast models, crop/agricultural models, solar energy forecast models, groundwater models, water usage and availability data, solar energy usage and availability data, or a combination thereof to the machine learning model for groundwater and solar energy usage optimization in a water and solar energy usage optimization system 430.

In one aspect, the machine learning component 406 may include a prediction component 408 for cognitively learning and predicting one or more weather forecast models, crop/agricultural models, solar energy forecast models, ground water models, water usage and availability data, solar energy usage and availability data, or a combination thereof in the water and solar energy usage optimization system 430. The machine learning component 406 may also include and/or use one or more data models representing data, weather forecast models, crop/agricultural models, solar energy forecast models, groundwater models, water usage and availability data, and/or solar energy usage and availability data. Additionally, the prediction component 408 may predict the amount of solar energy available for the agricultural region, predict the amount of water required for usage in the agricultural region, predict the excess solar energy to sell to a power grid, and/or predict the excessive water for non-agricultural usages.

The supply and demand forecast component 410 may predict the amount of water required for usage in the agricultural region (e.g., water required for watering crops on a farm). The supply and demand forecast component 410 may also predict (a) the amount of solar energy available in the agricultural region and (b) the amount of water available from rainfall and groundwater pumping.

The integrator component 440 may collect the predicted results from the supply and demand forecast component 410 and determine an amount of solar energy (e.g., PV energy) required and needed such as, for example, the amount of PV energy to pump water in a PV energy water pumping system (e.g., PV energy water pump 475).

The market connector component 445 may be used to facilitate, coordinate, and/or broker the sale of any excess solar energy in the agricultural region to a power grid. The market connector component 445 may be used to facilitate, coordinate, and/or broker the use of excessive water for non-agricultural usages.

Additionally, the market connector component 445 may be used to enable and drive user interaction where input may be required or received. That is, the market connector component 445 may send and receive (e.g., from device 420) information that may identify one or more opportunities (e.g., excessive water above a threshold may be used for consumption or use for community, public or private entities such as, for example, providing water for recreational services, governmental services, emergency response (e.g., fire services), building or constructing communities, and/or sales opportunities to potential buyers) to use the excessive water for non-agricultural usages and sell the excessive solar energy to a power grid. For example, the market connector component 445 may communicate to device 420 one or more messages.

The device 420 may include a graphical user interface (GUI) 422 enabled to display on the device 420 one or more user interface controls for a user to interact with the GUI 422. For example, the GUI 422 may display an interactive dialog with questions and/or answers to facilitate, coordinate, and/or broker the sale of excessive solar energy to a power grid and/or use the excessive water for non-agricultural usages. For example, the GUI 422 may indicate or display audibly and/or visually a message such as, for example, “There is a detected excessive amount of solar energy and water supply (for the agricultural region). Would you like to sell the solar energy to a power grid and use the excess water for non-agricultural purposes?”

Returning again to the machine learning component 406, the machine learning component 406 may apply one or more heuristics and machine learning based models using a wide variety of combinations of methods, such as supervised learning, unsupervised learning, temporal difference learning, reinforcement learning and so forth. Some non-limiting examples of supervised learning which may be used with the present technology include AODE (averaged one-dependence estimators), artificial neural network, backpropagation, Bayesian statistics, naive bays classifier, Bayesian network, Bayesian knowledge base, case-based reasoning, decision trees, inductive logic programming, Gaussian process regression, gene expression programming, group method of data handling (GMDH), learning automata, learning vector quantization, minimum message length (decision trees, decision graphs, etc.), lazy learning, instance-based learning, nearest neighbor algorithm, analogical modeling, probably approximately correct (PAC) learning, ripple down rules, a knowledge acquisition methodology, symbolic machine learning algorithms, sub symbolic machine learning algorithms, support vector machines, random forests, ensembles of classifiers, bootstrap aggregating (bagging), boosting (meta-algorithm), ordinal classification, regression analysis, information fuzzy networks (IFN), statistical classification, linear classifiers, fisher's linear discriminant, logistic regression, perceptron, support vector machines, quadratic classifiers, k-nearest neighbor, hidden Markov models and boosting. Some non-limiting examples of unsupervised learning which may be used with the present technology include artificial neural network, data clustering, expectation-maximization, self-organizing map, radial basis function network, vector quantization, generative topographic map, information bottleneck method, IBSEAD (distributed autonomous entity systems based interaction), association rule learning, apriori algorithm, eclat algorithm, FP-growth algorithm, hierarchical clustering, single-linkage clustering, conceptual clustering, partitional clustering, k-means algorithm, fuzzy clustering, and reinforcement learning. Some non-limiting examples of temporal difference learning may include Q-learning and learning automata. Specific details regarding any of the examples of supervised, unsupervised, temporal difference or other machine learning described in this paragraph are known and are considered to be within the scope of this disclosure.

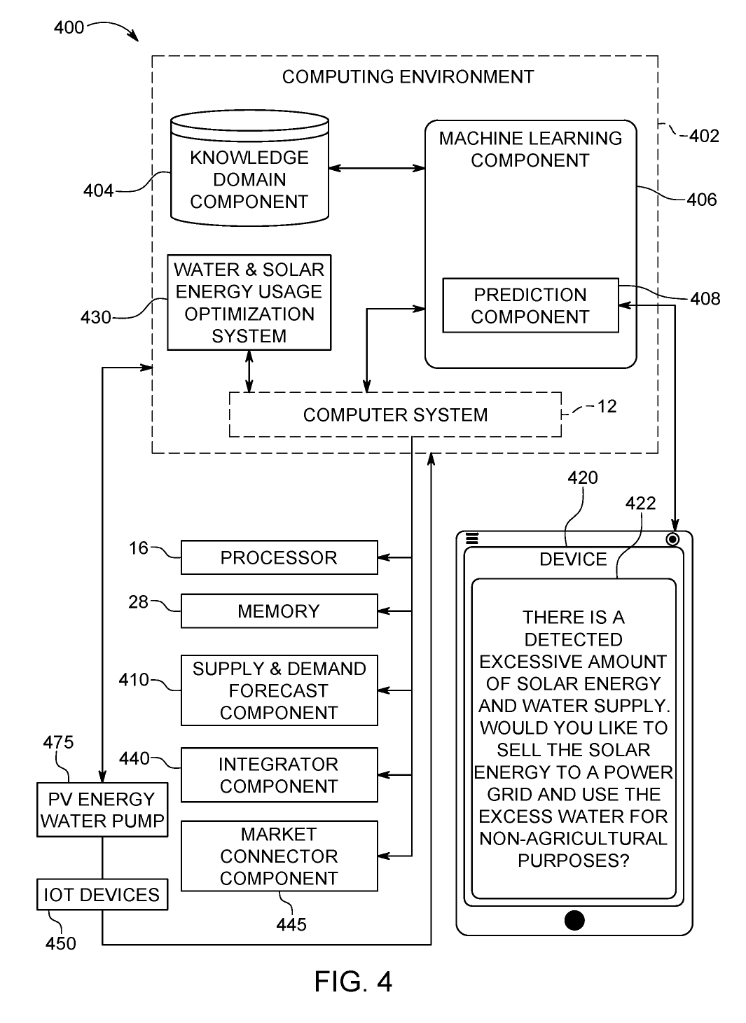

FIG. 5 is a simplified block/flow diagram illustrating certain aspects of functionality, or functional blocks 500, according to some embodiments of the present invention. As shown, a farm management strategy 502 may be used to compute and/or determine a crop model, at block 504. The crop model from 504 may be used to compute and/or determine an amount of water required for use in an agricultural region (e.g., a farm), as in block 506.

A weather forecast model 508 may be used to compute both a weather forecast (e.g., rain forecast), at block 512, and also a photovoltaics (PV) energy forecast model, at block 514. The PV forecast model from block 514 may move to block 522.

The amount of water required determined from block 506 and the rain forecast from block 512 may be sent to block 516, where it is determined whether or not there is a sufficient amount of water (e.g., greater than zero) to pump in a water pumping system based on the computed water required and rain forecast. If there is a sufficient amount of water at block 516, a groundwater model 518 may be used to compute and/or determine an amount of power (e.g., photovoltaics “PV” energy) required to pump groundwater to be used in the agricultural region, at block 520. The determined amount of required power from block 520 may move to block 522.

If there is not a sufficient amount of water at block 516 and in conjunction with the PV forecast model from block 514 and the determined amount of PV energy required to pump water from block 520, a determination operation may be performed to determine if there is an excess amount of PV energy available to sell to a power grid, as in block 522.

Although not shown in FIG. 5, an indication of whether or not groundwater theft (or over-discharge) is occurring may be generated and provided to a user (e.g., an authority monitoring the groundwater discharge) in any suitable manner. For example, the indication may be provided by electronic messages (e.g., text message, email, etc.), visual messages (e.g., on display screens), and/or aural messages (e.g., recorded messages, buzzers, etc.).

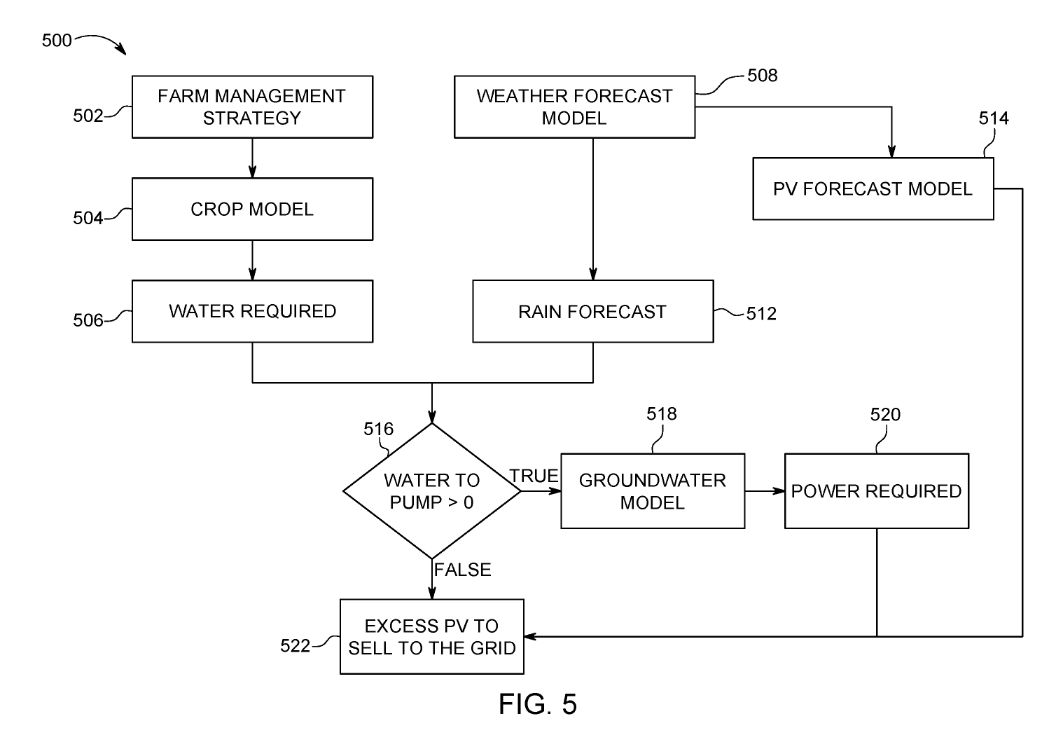

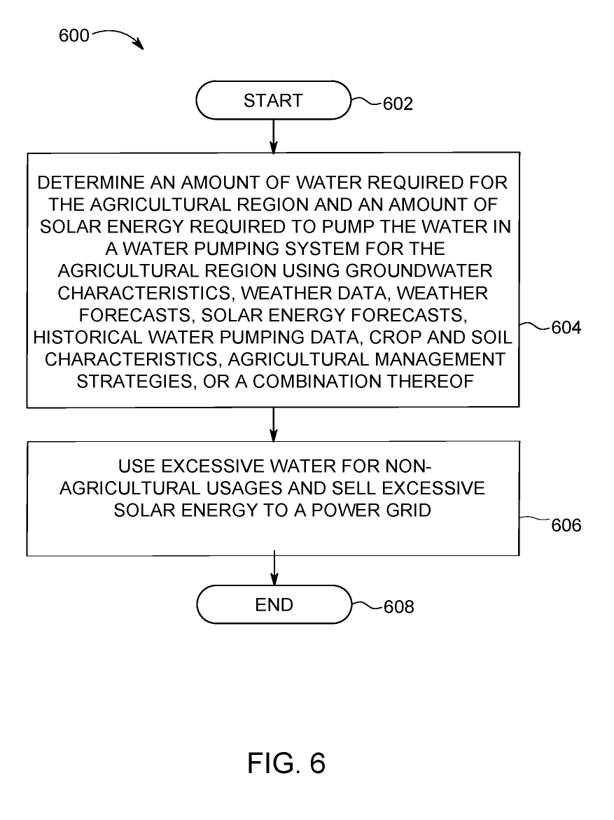

Turning now to FIG. 6, a method 600 for monitoring groundwater discharge using a processor is depicted, in which various aspects of the illustrated embodiments may be implemented. The functionality 600 may be implemented as a method executed as instructions on a machine, where the instructions are included on at least one computer readable medium or one non-transitory machine-readable storage medium. In one aspect, the functionality, operations, and/or architectural designs of FIGS. 1-4 may be implemented all and/or in part in FIG. 6.

The functionality 600 may start in block 602. An amount of water required for the agricultural region and an amount of solar energy required to pump the water in a water pumping system may be determined for the agricultural region using groundwater characteristics, weather data, weather forecasts, solar energy forecasts, historical water pumping data, crop and soil characteristics, agricultural management strategies, or a combination thereof, as in block 604. Excessive water may be used for non-agricultural usages and excessive solar energy may be sold to a power grid (according to the determining of block 604), as in block 606. Also, one or more opportunities to use excessive water for non-agricultural usages and excessive solar energy to sell to a power grid may be identified. The functionality 600 may end, as in block 608.

In one aspect, in conjunction with and/or as part of at least one block of FIGS. 5-6, the operations of 500 and/or 600 may include each of the following. The operations of 500 and/or 600 may determine the amount of water by measuring rainfall based on one or more IoT sensor devices at one of the plurality of locations in the agricultural region and groundwater discharge for at least one of the plurality of locations in the agricultural region based on measured groundwater heads.

The operations of 500 and/or 600 may predict the amount of solar energy available for the agricultural region, predict the amount of water required for usage in the agricultural region, predict the excess solar energy to sell to a power grid, and/or predict the excessive water for non-agricultural usages. An amount of photovoltaics (PV) energy required to pump the water pumping system may be determined according to water and solar energy supplies and demands in the agricultural region.

The operations of 500 and/or 600 may further continuously sample water usage and determine solar energy amounts over a selected time period by the one or more IoT sensors. A machine learning mechanism may be initialized using the feedback information from the one or more IoT sensors to predict water usage and solar energy generation.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowcharts and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowcharts and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowcharts and/or block diagram block or blocks.

The flowcharts and block diagrams in the Figures illustrate the architecture, functionality, and operation of possible implementations of systems, methods, and computer program products according to various embodiments of the present invention. In this regard, each block in the flowcharts or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts or carry out combinations of special purpose hardware and computer instructions.

1. A method for groundwater and solar energy usage optimization for an agricultural region in an Internet of Things (IoT) computing environment by one or more processors, comprising:
determining an amount of water required for the agricultural region and an amount of solar energy required to pump the water in a water pumping system for the agricultural region according to groundwater characteristics, weather data, weather forecasts, solar energy forecasts, historical water pumping data, crop and soil characteristics, agricultural management strategies, or a combination thereof.

2. The method of claim 1, further including determining the amount of water by measuring rainfall based on one or more IoT sensor devices at one of the plurality of locations in the agricultural region and groundwater discharge for at least one of the plurality of locations in the agricultural region based on measured groundwater heads.

3. The method of claim 1, further including:
predicting the amount of solar energy available for the agricultural region; and
predicting the amount of water required for usage in the agricultural region.

4. The method of claim 1, further including:
predicting the excess solar energy to sell to a power grid; and
predicting the excessive water for non-agricultural usages.

5. The method of claim 1, further including determining an amount of photovoltaics (PV) energy required to pump the water pumping system according to water and solar energy supplies and demands in the agricultural region.

6. The method of claim 1, further including continuously sampling water usage and determining solar energy amounts over a selected time period by the one or more IoT sensors.

7. The method of claim 1, further including initializing a machine learning mechanism using the feedback information from the one or more IoT sensors to predict water usage and solar energy generation.

8. A system for groundwater and solar energy usage optimization for an agricultural region in an Internet of Things (IoT) computing environment, comprising:
one or more computers with executable instructions that when executed cause the system to:
determine an amount of water required for the agricultural region and an amount of solar energy required to pump the water in a water pumping system for the agricultural region according to groundwater characteristics, weather data, weather forecasts, solar energy forecasts, historical water pumping data, crop and soil characteristics, agricultural management strategies, or a combination thereof.

9. The system of claim 8, wherein the executable instructions further determine the amount of water by measuring rainfall based on one or more IoT sensor devices at one of the plurality of locations in the agricultural region and groundwater discharge for at least one of the plurality of locations in the agricultural region based on measured groundwater heads.

10. The system of claim 8, wherein the executable instructions further:
predict the amount of solar energy available for the agricultural region; and
predict the amount of water required for usage in the agricultural region.

11. The system of claim 8, wherein the executable instructions further:
predict the excess solar energy to sell to a power grid; and
predict the excessive water for non-agricultural usages.

12. The system of claim 8, wherein the executable instructions further determine an amount of photovoltaics (PV) energy required to pump the water pumping system according to water and solar energy supplies and demands in the agricultural region.

13. The system of claim 8, wherein the executable instructions further continuously sample water usage and determine solar energy amounts over a selected time period by the one or more IoT sensors.

14. The system of claim 8, wherein the executable instructions further initialize a machine learning mechanism using the feedback information from the one or more IoT sensors to predict water usage and solar energy generation.

15. A computer program product for groundwater and solar energy usage optimization for an agricultural region in an Internet of Things (IoT) computing environment by a processor, the computer program product comprising a non-transitory computer-readable storage medium having computer-readable program code portions stored therein, the computer-readable program code portions comprising:
an executable portion that determines an amount of water required for the agricultural region and an amount of solar energy required to pump the water in a water pumping system for the agricultural region according to groundwater characteristics, weather data, weather forecasts, solar energy forecasts, historical water pumping data, crop and soil characteristics, agricultural management strategies, or a combination thereof.

16. The computer program product of claim 15, further including an executable portion that determines the amount of water by measuring rainfall based on one or more IoT sensor devices at one of the plurality of locations in the agricultural region and groundwater discharge for at least one of the plurality of locations in the agricultural region based on measured groundwater heads.

17. The computer program product of claim 15, further including an executable portion that:
predicts the amount of solar energy available for the agricultural region;
predicts the amount of water required for usage in the agricultural region;
predicts the excess solar energy to sell to a power grid; and
predicts the excessive water for non-agricultural usages.

18. The computer program product of claim 15, further including an executable portion that determines an amount of photovoltaics (PV) energy required to pump the water pumping system according to water and solar energy supplies and demands in the agricultural region.

19. The computer program product of claim 15, further including an executable portion that continuously samples water usage and determines solar energy amounts over a selected time period by the one or more IoT sensors.

20. The computer program product of claim 15, further including an executable portion that initializes a machine learning mechanism using the feedback information from the one or more IoT sensors to predict water usage and solar energy generation.
